(19) United States
(12) Patent Application Publication
(10) Pub. No.: US 2023/0346684 A1
(43) Pub. Date: Nov. 2, 2023

(54) PIGMENT DISPERSIONS COMPRISING POLYETHER POLYMERS FOR COSMETICS

(71) Applicants: P2 SCIENCE, INC., Woodbridge, CT (US); AMERICAN COLORS, INC., Sandusky, OH (US)

(72) Inventors: Madison PICCIRILLO, Sheffield Lake, OH (US); Kayla EYER, Sandusky, OH (US); Adalberto FLEITAS, Norwalk, CT (US); Chloe HENNINGER, West Hartford, CT (US)

(21) Appl. No.: 18/311,076

(22) Filed: May 2, 2023

Related U.S. Application Data
(60) Provisional application No. 63/337,568, filed on May 2, 2022.

(51) Int. Cl. A61K 8/86; A61K 8/29; A61K 8/26; A61K 8/19; A61Q 1/02

(52) U.S. Cl. CPC A61K 8/86; A61K 8/29; A61K 8/26; A61K 8/19; A61Q 1/02; A61K 2800/43; A61K 2800/34

(57) ABSTRACT

The invention relates to pigment dispersions for finished cosmetic and personal care compositions, the pigment dispersions comprising pigment dispersed in certain citronellol-based polyether polymers and their derivatives, and methods of making and using the same.

### CROSS-REFERENCE TO RELATED APPLICATIONS

This application is U.S. national application filed under 35 U.S.C. § 111(a) which claims priority to, and the benefit of, U.S. Provisional Application Ser. No. 63/337,568, filed on May 2, 2022, the contents of which are hereby incorporated by reference in its entirety.

### FIELD

The invention relates to pigment dispersions for finished cosmetic and personal care compositions, the pigment dispersions comprising pigment dispersed in certain citronellol-based polyether polymers and their derivatives, and methods of making and using the same. In some embodiments, the polyether polymers serve as a grinding vehicle and/or carrier for an untreated, uncoated pigment. Preferably, the pigment dispersion provides a color concentrate having an NOI (natural origin index) value of close to 1.

### BACKGROUND

Personal care compositions are compositions suitable for topical application to the human body, such as the skin and hair, for improving appearance and/or cleanliness. Examples of personal care compositions include skin care products (e.g., facial creams, moisturizers, face and body lotions, sunscreens, foundation, mascara, eye-liner, lipsticks, liquid soaps, solid soaps, body washes, cleansers, and the like) and hair care products (e.g., shampoos, conditioners, styling gels and hairsprays). These compositions are often intended to clean and/or to moisturize the skin and hair, and keep them in a smooth condition.

Personal care compositions must be carefully formulated to provide maximum wear and effect, and to avoid incompatibilities between ingredients which can affect stability, storability, and appearance.

Cosmetics and other similar products are commonly strongly colored. Critical to the formulation of a cosmetic composition, and other personal care compositions, is the delivery vehicle used for incorporating coloring agents, such as pigments, into the compositions. It is important that the delivery system provide for stability of the color and consistency in color between batches of product, preferably also with a minimum amount of manufacturing time and labor required. It has also become very popular to use naturally derived ingredients in such compositions.

Liquid colorants, or dispersions, as pigment delivery vehicles have been used for years across many industries, including cosmetics and personal care, as well as coatings, sealants, adhesives, and plastics. Essentially, a dispersion wets out a pigment, stabilizing it in a liquid carrier, also called the grinding vehicle. This can sometimes be achieved by simply mixing the pigment into the grinding vehicle, where the grinding vehicle has enough dispersive properties to stabilize the solid particles itself. Otherwise, dispersants, also called surfactants, are necessary to achieve stabilization. Once the pigment is incorporated into the liquid carrier, with or without dispersants, it can then be grinded through mechanical action, for example in a media mill or three-roll mill (commonly used for color cosmetics). Grind is achieved when the pigment is the desired particle size.

Several factors are considered crucial in evaluating the performance of a pigment dispersion: wet-out time (fast wet-out being preferable), ability to undergo high-speed grinding, the amount of milling required to achieve the desired particle size and color strength; and the final viscosity of the dispersion.

Pigment wet-out is defined as the time it takes for a dry pigment to be incorporated into a liquid vehicle. A poor wet-out would display as pigment sitting on top of the liquid carrier despite high-speed mixing. In this scenario, the operator must add pigment slowly to avoid choking the mixer. The operator is required to monitor the mixer until the pigment is incorporated. For a poor wet-out, the pigment would require more time and mechanical action to mix, even before grinding can begin. An excellent wet-out would look similar to a simple dilution, where the pigment immediately mixes into the vehicle with very little time spent sitting on the surface. This would mean that the dispersion requires less pre-mixing time and operator monitoring.

Pigment color can be heavily dependent on particle size and processing. Pigment strength is typically maximized at a certain particle size, with additional processing being detrimental to the color strength, or else moving the color space in an entirely new area, with no opportunity for recovery. Because of this, it is crucial to disperse pigment in a grinding vehicle that allows for quick and accurate pigment grinding.

Final viscosity is more dependent on pigment and carrier chemical properties than dispersion quality. If a pigment is not properly dispersed, the dispersion may appear lumpy, or change viscosity over time due to pigment agglomeration or settling.

Traditional liquid polymers, such as polyethylene glycols, mixed glycol polymers, poloxamers, and silicone polymers, have important utility in cosmetic and personal care applications. For example, they can be used as emulsifiers, preservatives, stabilizers, fragrance carriers, fragrance retention agents, fragrance fixers, anti-malodor agents, anti-foaming agents, lubricants, emollients, surfactants, or as protective barriers for skin healing and UV protection, and as a substitute for petroleum-based white oil (a mixture of alkanes and cycloalkanes).

Silicones, in particular, are widely used in personal care and cosmetics products for their sensory properties. Silicones are also utilized for their hydrophobic properties, imparting water resistance to finished cosmetics. They can also be relied upon for their spreading ability, film-forming ability, skin feel, volatility and permeability. Recently, however, the cosmetics and personal care markets have been moving away from silicones and other synthetic ingredients. This is largely due to a consumer push towards “natural” or naturally-derived ingredients. As these types of products become more in-demand, cosmetics companies are forced to find natural alternatives to commonly used synthetic raw materials.

Some natural oils that have been studied as silicone replacements include castor oil and other vegetable oils. However, many of these suffer from several drawbacks, such as an inability to match silicone's sensory feel and performance properties, and inability to handle high pigments loadings (e.g., above 50 wt. %) without significant adverse effects on viscosity. Pigment dispersions made using these oils often also suffer from an undesired color contribution from the oil itself, such as a yellow tint, which is also often highly variable between different batches of such oils. Such natural oils therefore make poor replacements for silicones.

Therefore, there still remains a need for new liquid polymer materials to replace silicones in cosmetic and personal are compositions, which can be produced in a facile manner, be easily derivatized to modify functions and properties, and which can preferably be made from safe and sustainable raw materials.

Citronellol is a naturally occurring molecule that is also commercially available on a large scale. WO 2016/033437, WO 2019/028053, US 2017/0283553, and US 2020/0165383, the contents of each of which are hereby incorporated by reference in their entireties, disclose novel polyether polymers derived from citronellol. The citronellol monomer is effectively polymerized in a controlled way to yield a number of well-characterized polymeric ether alcohol products. In addition, as these polymers as initially formed may possess primary alcohol functional groups, WO 2019/028053 further discloses functionalization of the alcohol to derive various ether, ester, and other derivative products. According to the nature of functionalization, chemical and physical properties (e.g., density, surface tension, refractive index, solubility, viscosity, hydrophilicity, hydrophobicity, etc.) of these polymers can be tuned appropriately for specific applications. Additional polycitronellol polymers and compositions comprising them are disclosed in WO 2021/133994, US 2021/0230364, WO 2021/178217, and US 2021/0275430, the contents of each of which are hereby incorporated by reference in their entireties.

The present disclosure provides new pigment dispersion formulations for cosmetic and personal care compositions comprising these polyether polymers, for example, as replacement for silicone polymers or other ethereal polymers in such compositions.

### BRIEF SUMMARY

In a surprising advancement in polymer science, the inventors' prior publications US 2017/0283553, US 2020/0165383, US 2021/0230364, and US 2021/0275430, the contents of each of which are incorporated herein by reference, have taught generally how to prepare polyether polymers and derivatives thereof. These polyethers represent an advance in liquid polymer technology and carry with them many desirable benefits for commercial fields of application.

The present disclosure builds on the inventors' prior work by providing new pigment dispersions for cosmetic and personal care compositions comprising such polymers.

Citronellol polyether polymers have a natural origin index (NOI) of 1 making them highly prized for use in cosmetic and personal care products. In addition, the inventors have discovered that these polymers are surprisingly superior as carrier vehicles for untreated pigments compared to silicones. In particular, pigment dispersions according to the present disclosure provide a decreased pigment wet-out time, decreased processing time to achieve the desired pigment grind, improved color strength, and processible viscosity, meaning a viscosity that can be mixed by a high-speed mixer. The present disclosure further provides a pigment dispersion which will improve customer perception of sustainability benefits of products made with the dispersions, and will reduce manufacturing time and cost, minimize raw material requirements, and improves the sensory properties of the resulting product compositions (providing comparable sensory properties to silicone-based dispersion, or improved sensory properties).

In a first aspect, the present disclosure provides a pigment dispersion comprising a pigment dispersed in a grinding vehicle and/or carrier, wherein the grinding vehicle and/or carrier comprises a compound according to Formula I below:

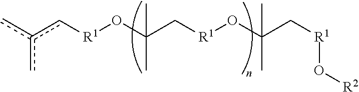

wherein R¹ is CH₂CH₂CH(CH₃)CH₂CH₂, R² is H, C₁₋₂₀alkyl (e.g., C₁₋₃alkyl), or —C(O)—C₁₋₂₀alkyl (e.g., —C(O)—C₁₋₃alkyl),
and wherein n is an integer between 0 and 20.
In some embodiments, n is an integer between 0 and 10 (e.g., 0 to 4).

In some embodiments, the grinding vehicle and/or carrier comprises a mixture of compounds according to Formula I, for example, a mixture of compounds that only vary in the integer n. In some embodiments, the mixture of compounds according to Formula I have a number average or weight average molecular weight, optionally exclusive of the group R², of 150 to 2000 Daltons (e.g., 300 to 800 Daltons), and/or a polydispersity (M_{w}/Mₙ) in the range of 1 to 5 (optionally without taking into account the group R²).

It is understood that [symbol] represents an optional double bond (i.e., either a single or double bond), and thus that the terminal group,

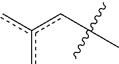

may have any one of the three indicated optional bonds present (i.e., a double bond) or all optional bonds absent (i.e., all single bonds).

In further embodiments, the present disclosure provides methods of making and/or using the pigment dispersions, such as, to make cosmetic or personal care compositions.

Additional features and advantages of the dispersions, composition, and methods disclosed herein will be apparent from the following detailed description.

### DETAILED DESCRIPTION

Although specific embodiments of the present disclosure will now be described with reference to the examples provided herein, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure, and as further defined in the appended claims.

Unless defined otherwise, all technical and scientific terms used herein have the same meanings as commonly understood by one of ordinary skill in the art to which this disclosure belongs.

In a first aspect, the present disclosure provides a pigment dispersion (Dispersion 1) comprising a pigment dispersed in a grinding vehicle and/or carrier, wherein the grinding vehicle and/or carrier comprises a compound according to Formula I, as defined herein above. In further embodiments of the first aspect, the present disclosure provides:
1.1 Dispersion 1, wherein in the compound of Formula I, R² is H.
1.2 Dispersion 1, wherein in the compound of Formula I, R² is C₁₋₂₀alkyl (e.g., lower alkyl (e.g., C₁₋₆), or C₁₋₁₂).
1.3 Dispersion 1, wherein in the compound of Formula I, R² is selected from methyl, ethyl, propyl, isopropyl, n-butyl, isobutyl, sec-butyl, tert-butyl, n-pentyl, n-hexyl, n-octyl, or n-decyl.
1.4 Dispersion 1, wherein in the compound of Formula I, R² is —C(O)—C₁₋₂₀ alkyl (e.g., —C(O)—C₁₋₆ alkyl).
1.5 Dispersion 1, wherein in the compound of Formula I, R² is —C(O)—C₁₋₆ alkyl, optionally wherein R² is —C(O)—C₁₋₅ alkyl, —C(O)—C₁₋₄ alkyl, —C(O)—C₁₋₃ alkyl or —C(O)—C₁₋₂ alkyl.
1.6 Dispersion 1, wherein in the compound of Formula I, R² is —C(O)—C₁₋₆ alkyl and said C₁₋₆ alkyl is selected from methyl, ethyl, propyl, isopropyl, n-butyl, isobutyl, sec-butyl, tert-butyl, n-pentyl, or n-hexyl.
1.7 Dispersion 1 or any of 1.1 et seq., wherein in the compound of Formula I, n is 0 to 10 (e.g., 1 to 9, or 2 to 8, or 1 to 7, or 2 to 6, or 1 to 5, or 2 to 4, etc.).
1.8 Dispersion 1 or any of 1.1 et seq., wherein in the compound of Formula I, n is 0 to 8 (e.g., 1 to 8, or 2 to 8, or 1 to 7, or 2 to 6, or 1 to 5, or 2 to 4, etc.).
1.9 Dispersion 1 or any of 1.1 et seq., wherein in the compound of Formula I, n is 1 to 8 (e.g., 1 to 7, or 2 to 6, or 1 to 5, or 2 to 5, or 1 to 4, or 2 to 4, etc.).
1.10 Dispersion 1 or any of 1.1 et seq., wherein in the compound of Formula I, n is 1 to 7 (e.g., 1 to 4, or 2 to 5, or 3 to 6, or 4 to 7, or 2 to 6).
1.11 Dispersion 1 or any of 1.1 et seq., wherein in the compound of Formula I, n is 0, 1, 2, 3, 4, 5, 6, 7, or any combination thereof.
1.12 Dispersion 1 or any of 1.1 et seq., wherein in the compound of Formula I, the terminal group

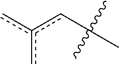

is

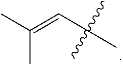 [SMILES: *C=C(C)C]

1.13 Dispersion 1 or any of 1.1 et seq., wherein in the compound of Formula I, the terminal group

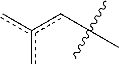

is

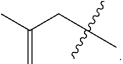 [SMILES: *CC(=C)C]

1.14 Dispersion 1 or any of 1.1 et seq., wherein in the compound of Formula I, the terminal group

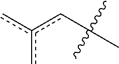

is

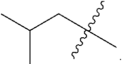 [SMILES: *CC(C)C]

1.15 Dispersion 1 or any of 1.1 et seq., the Compound of Formula I is:

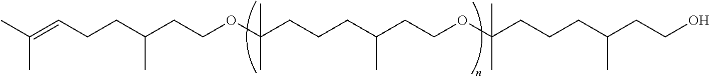

where n: 0-20 (e.g., 0-10, 1-9, 1-7, 1-4, 1-3, 0-5, 0-4, 0-3, 0, 1, 2, 3, 4, or 5).
1.16 Compound 1 or any of 1.1 et seq., wherein the compound of Formula I is enantiomerically enriched, e.g., having a diastereomeric excess (e.g., an enantiomeric excess (e.e.)) of greater than 70%.
1.17 Dispersion 1.16, wherein the compound of Formula I has a diastereomeric excess (e.g., an enantiomeric excess (e.e.)) of greater than 80%, or greater than 85%, or greater than 90%, or greater than 95%, or greater than 97% or greater than 99%.
1.18 Dispersion 1.16 or 1.17, wherein each chiral carbon of the compound of Formula I, exclusive of the group R², has the (R)-configuration, optionally wherein each chiral carbon of the group R² also has the (R)-configuration.
1.19 Dispersion 1.16 or 1.17, wherein each chiral carbon of the compound of Formula I, exclusive of the group R², has the (S)-configuration, optionally wherein each chiral carbon of the group R² also has the (S)-configuration.
1.20 Dispersion 1.16 or 1.17, wherein each chiral carbon of the compound of Formula I, exclusive of the group R², has the (R)-configuration, and wherein each chiral carbon of the group R² has the (S)-configuration.
1.21 Dispersion 1.16 or 1.17, wherein each chiral carbon of the compound of Formula I, exclusive of the group R², has the (S)-configuration, and wherein each chiral carbon of the group R² has the (R)-configuration.
1.22 Dispersion 1 or any of 1.1 et seq., wherein the dispersion comprises a single compound of Formula I according to any one of the compounds described in Dispersion 1 or 1.1-1.22, the compound being present in an amount of 0.1 to 50% by weight of the dispersion, e.g., 0.1-40%, or 0.1-30%, or 0.1-20%, or 0.1-10%, or 0.1-5%, or 1-40%, or 1-30%, or 1-20%, or 1-10%, or 1-5%, or about 3.5%, or 5-15%, e.g., about 10%, or 30-50%, or 30-40%.
1.23 Dispersion 1 or any of 1.1 et seq., wherein the dispersion comprises one or more compounds of Formula I (e.g., from one up to ten specific compounds), wherein each compound is independently a compound described in Dispersion 1 or any one of 1.1-1.22, each compound being present in an amount of 0.01 to 40% by weight of the dispersion, e.g., 0.01-30%, or 0.01-20%, or 0.01-10%, or 0.01-5%, or 0.01-1%, or 0.01-0.1%, or 0.1-20%, or 0.1-15%, or 0.1-10%, or 0.1-5%, or 0.1 to 1%.
1.24 Dispersion 1 or any of 1.1 et seq., wherein the dispersion comprises a mixture of compounds according to Formula I.
1.25 Dispersion 1.24, wherein the mixture of compounds according to Formula I vary only in the integer n.
1.26 Dispersion 1.24 or 1.25, wherein the mixture of compounds according to Formula I have a number average molecular weight (Mₙ), optionally exclusive of the group R², of 150 to 2000 Daltons (e.g., 300 to 800 Daltons).
1.27 Dispersion 1.26, wherein the mixture of compounds according to Formula I have a number average molecular weight (Mₙ), optionally exclusive of the group R², of 300 to 1900 Daltons, e.g., 300 to 1600 Daltons, or 300 to 1300 Daltons, or 300 to 1100 Daltons, or 300 to 1000 Daltons, or 300 to 800 Daltons, or 300 to 700 Daltons, or 300 to 500 Daltons, or 400 to 1000 Daltons, or 400 to 700 Daltons, or 600 to 1100 Daltons, or 600 to 1000 Daltons, or 600 to 800 Daltons or about 500 Daltons, or about 414 Daltons.
1.28 Dispersion 1.26 or 1.27, wherein the mixture of compounds according to Formula I have a weight average molecular weight (M_{w}), optionally exclusive of the group R², of 150 to 2000 Daltons (e.g., 300 to 800 Daltons).
1.29 Dispersion 1.28, wherein the weight average molecular weight (M_{w}) of the compounds in the dispersion, optionally exclusive of the group R², is 300 to 1900 Daltons, e.g., 300 to 1600 Daltons, or 300 to 1300 Daltons, or 300 to 1100 Daltons, or 300 to 1000 Daltons, or 300 to 800 Daltons, or 300 to 700 Daltons, or 300 to 500 Daltons, or 400 to 1000 Daltons, or 400 to 700 Daltons, or 600 to 1100 Daltons, or 600 to 1000 Daltons, or 600 to 800 Daltons, or about 438 Daltons.
1.30 Any of Dispersions 1.24-1.29, wherein the mixture of compounds according to Formula I have a polydispersity (M_{w}/Mₙ) in the range of 1 to 5 (optionally without taking into account the group R²).
1.31 Dispersion 1.30, wherein the polydispersity (M_{w}/Mₙ) is in the range of 1 to 4, or 1 to 3, or 1 to 2.5, or 1 to 2, or 1 to 1.5, or about 1 to 1.25, or about 1, or 1.5 to 3.5, or 1.5 to 2.5, or about 1.5, or 2 to 4, or 2 to 3, or 2 to 2.5, or about 2, or 1 to 1.25, or 1 to 1.20, or 1 to 1.15, or 1 to 1.10, or about 1.06.
1.32 Any of Dispersions 1.24-1.31, wherein the compounds of Formula I in the dispersion have an average n value of 1 to 7, measured either as a weight average or number average.
1.33 Any of Dispersions 1.24-1.31, wherein the compounds of Formula I in the dispersion have an average n value of 0 to 8, measured either as a weight average or number average.
1.34 Any of Dispersions 1.24-1.33, wherein the compounds of Formula I in the dispersions are all compounds of the structure:

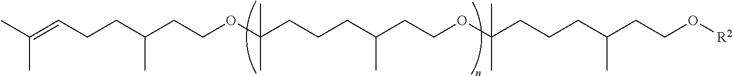

wherein R2 is H and n is from 1 to 7.
1.35 Dispersion 1.34, wherein at least 95% of the compounds of Formula I by weight have an n value of 1, 2, 3, 4, 5, 6 or 7, e.g., at least 97% or at least 98% or at least 99% of said compounds.
1.36 Dispersion 1.34 or 1.35, wherein at least 90% of the compounds of Formula I by weight have an n value of 1, 2, 3, 4, 5, 6 or 7, e.g., 90-98% or 95-98% of said compounds.
1.37 Dispersion 1.34, 1.35 or 1.36, wherein at least 70% of the compounds of Formula I by weight have an n value of 1, 2, 3, 4, 5, 6 or 7, e.g., at least 80%, or 80-90% or 80-85% of said compounds.
1.38 Dispersion 1.34, 1.35, 1.36 or 1.37, wherein at least 30% of the compounds of Formula I by weight have an n value of 0, e.g., at least 40%, or 40-60% or 45-55% of said compounds.
1.39 Dispersion 1, or any of 1.1-1.38, wherein the dispersion is a non-aqueous dispersion.
1.40 Dispersion 1, or any of 1.1-1.39, wherein the grinding vehicle and/or carrier further comprises one or more natural or naturally-derived oils, e.g., vegetable oils (e.g., castor oil).
1.41 Dispersion 1, or any of 1.1-1.39, wherein the grinding vehicle and/or carrier consists of the one or more Compounds of Formula I as described hereinabove.
1.42 Dispersion 1, or any of 1.1-1.41, wherein the pigment is dispersed homogenously in the grinding vehicle and/or carrier.
1.43 Dispersion 1, or any of 1.1-1.42, wherein the one or more Compounds of Formula I act as a grinding vehicle, as a carrier, or as a grinding vehicle and as a carrier.
1.44 Dispersion 1, or any of 1.1-1.43, wherein the pigment is selected from iron oxide (e.g., red iron oxide, yellow iron oxide, black iron oxide or a mixture thereof), titanium dioxide, ultramarines, mica, carbon black (e.g., Black 2), or any combination thereof.
1.45 Dispersion 1, or any of 1.1-1.44, wherein the pigments are untreated and/or uncoated, and optionally wherein the pigments are cosmetic-grade.
1.46 Dispersion 1, or any of 1.1-1.45, wherein the dispersion further comprises a dispersant.
1.47 Dispersion 1.46, wherein the dispersant is selected from polyhydroxystearic acid, bis-hydroxyethyl aminoethyl maleated soybean oil, polysorbate 80, sorbitan monooleate, PEG-4 laurate, PEG-141aurate, or any combination thereof (e.g., polysorbate 80 and sorbitan monooleate).
1.48 Dispersion 1, or any of 1.1-1.47, wherein the dispersion does not comprise any silicone polymer.
1.49 Dispersion 1, or any of 1.1-1.48, wherein the dispersion is free of any one or more of: surfactants, emulsifiers, emollients, humectants, preservatives, stabilizers, rheological additives, antioxidants, thickeners, and hydrocarbons.
1.50 Dispersion 1, or any of 1.1-1.49, wherein the dispersion consists essentially of the pigment or pigments, the compound or compounds of Formula I, and optionally the dispersant.
1.51 Dispersion 1, or any of 1.1-1.50, wherein the dispersion comprises 40% or more of the pigment or pigments, by weight of the dispersion, e.g., 50% or more, or 60% or more, or 70% or more, or 80% or more, or 90% or more, or 95% or more.
1.52 Dispersion 1, or any of 1.1-1.50, wherein the dispersion comprises 40 to 80% of the pigment or pigments, by weight of the dispersion, e.g., 45 to 80%, or 50 to 80%, or 55 to 80%, or 60 to 80%, or 65 to 80%, or 70 to 80%, or 75 to 80%, or 40 to 70%, or 45 to 70%, or 50 to 70%, or 55 to 70%, or 60% to 70%, or 55 to 65%, or about 60%.
1.53 Dispersion 1, or any of 1.1-1.50, wherein the dispersion comprises 5% or more of the pigment or pigments, by weight of the dispersion, e.g., 10% or more, or 20% or more, or 30% or more.
1.54 Dispersion 1, or any of 1.1-1.50, wherein the dispersion comprises 5 to 40% of the pigment or pigments, by weight of the dispersion, e.g., 5 to 35%, or 5 to 30%, or 5 to 25%, or 5 to 20%, or 5 to 15%, or 5 to 10%, or 5 to 25%, or 5 to 20%, or 5 to 15%, or 10 to 40%, or 10 to 30%, or 10% to 20%.
1.55 Dispersion 1, or any of 1.1-1.54, wherein the dispersion comprises 50% or less of the compound or compounds of Formula I, by weight of the dispersion, e.g., 40% or less, or 30% or less, or 20% or less, or 10% or less, or 5% or less.
1.56 Dispersion 1, or any of 1.1-1.54, wherein the dispersion comprises 10 to 50% of the compound or compounds of Formula I, by weight of the dispersion, e.g., 15 to 50%, or 20 to 50%, or 25 to 50%, or 30 to 50%, or 35 to 50%, or 40 to 50%, or 15 to 40%, or 20 to 40%, or 25 to 40%, or 30 to 40%, or 35 to 40%, or about 37%.
1.57 Dispersion 1, or any of 1.1-1.54, wherein the dispersion comprises 90% or less of the compound or compounds of Formula I, by weight of the dispersion, e.g., 85% or less, or 80% or less, or 75% or less, or 70% or less, or60% or less.
1.58 Dispersion 1, or any of 1.1-1.54, wherein the dispersion comprises 50-90% of the compound or compounds of Formula I, by weight of the dispersion, e.g., 55 to 90%, or 60 to 90%, or 65 to 90%, or 70 to 90%, or 60 to 80%, or 65 to 80%, or 70 to 80%.
1.59 Dispersion 1, or any of 1.1-1.58, wherein the dispersion comprises not more than 25% by weight of any dispersant(s) or other components (other than the pigment(s) and the compound(s) of Formula I), by weight of the dispersion, e.g., not more than 20%, or not more than 15%, or not more than 10%, or not more than 5%, or not more than 4%, or not more than 3%, or not more than 2%, or not more than 1%.
1.60 Dispersion 1, or any of 1.1-1.58, wherein the dispersion comprises 1 to 20% by weight of any dispersant(s), by weight of the dispersion, e.g., 1 to 15%, or 1 to 10%, or 1 to 5%, or 1 to 4%, or 1 to 3%, or 1 to 2%, or 5-20%, or 5-15%, or 5-10%, or 10-15%, or 2 to 15%, or 2 to 10%, or 2 to 8%, or 2 to 6%, or 2 to 5%, or 2 to 4% or 2 to 3%, or about 4 to 6%, or about 6%.
1.61 Dispersion 1, or any of 1.1-1.61, wherein the dispersion comprises 40-80% pigment (e.g., 55-65%, or about 60%), and 20-60% of one or more compounds of Formula I (e.g., 30-50%, or 35-45%, or about 38%), and optionally 1-6% of a dispersant (e.g., 2-5%, or 2-4%, or 2-3%), optionally wherein the dispersant is bis-hydroxyethyl aminoethyl maleated soybean oil, and optionally wherein the pigment is an iron oxide, and optionally wherein the one or more compounds of Formula I are compounds according to formula 1.34.
1.62 Dispersion 1, or any of 1.1-1.61, wherein the dispersion comprises 10-30% pigment (e.g., 10-20%, or about 13%), and 60-90% of one or more compounds of Formula I (e.g., 70-80%, or about 75%), and optionally 5-20% of a dispersant (e.g., 10-20%, or 10-15%, or 13%), optionally wherein the dispersant is polysorbate 80, sorbitan oleate, or a mixture thereof, and optionally wherein the pigment is carbon black, and optionally wherein the one or more compounds of Formula I are compounds according to formula 1.34.
1.63 Dispersion 1, or any of 1.1-1.62, wherein the pigment is milled to a grind greater than or equal to a Hegman grind of 6, e.g., a Hegman grind of 6 to 8, or 6 to 7, or 7 to 8, or about 6, or about 7, or about 8.
1.64 Dispersion 1, or any of 1.1-1.63, wherein the dispersion is homogenous, e.g., the dispersion is high-sped to achieve homogenization.
1.65 Dispersion 1, or any of 1.1-1.64, wherein the one or more pigments each disperse in the compound or compounds of formula I without mixing or with minimal mixing necessary to achieve homogeneity.
1.66 Dispersion 1, or any of 1.1-1.65, wherein the dispersion is physically stable, e.g., the dispersion remains homogenous without the formation of phases, precipitates, agglomerates, lumps, or other inhomogeneities, and/or, the viscosity of the dispersion does not change significantly (e.g., less than 5%).
1.67 Dispersion 1, or any of 1.1-1.66, wherein the dispersion has a pigment wet out time of less than 250 seconds, or less than 150 seconds, or less than 100 seconds, or less than 85 seconds, or less than 60 seconds, or less than 45 seconds, or less than 35 seconds, or less than 25 seconds.
1.68 Dispersion 1, or any of 1-1.1.67, wherein the dispersion comprises or consists of:
   (a) 50-70% titanium dioxide (e.g., about 60%), 20-50% compounds(s) of Formula I (e.g., about 34%), and 0-10% dispersant (e.g., PHSA, e.g., about 0% or about 6%), by weight of the dispersion;
   (b) 50-70% yellow iron oxide (e.g., about 61%), 20-50% compounds(s) of Formula I (e.g., about 39%), and 0-10% dispersant (e.g., PHSA, e.g., about 0% or about 6%), by weight of the dispersion;
   (c) 50-70% red iron oxide (e.g., about 61%), 20-50% compounds(s) of Formula I (e.g., about 37%), and 0-10% dispersant (e.g., PHSA, e.g., about 0% or about 2.4%), by weight of the dispersion; or
   (d) 50-70% black iron oxide (e.g., about 60%), 20-50% compounds(s) of Formula I (e.g., about 38%), and 0-10% dispersant (e.g., MSO, e.g., about 0% or about 2.4%), by weight of the dispersion.
1.69 Dispersion 1, or any of 1.1-1.68, wherein the pigment is the only ingredient in the dispersion which has a color affecting the color of the dispersion (e.g., all other ingredients are colorless or do not impart any effect on the color of the dispersion).
1.70 Dispersion 1, or any of 1.1-1.69, wherein the dispersion achieves a Hegman grind of at least 4 after one hour of high-speed mixing, e.g., a Hegman grind of at least 5 or at least 6, after one hour of high-speed mixing.
1.71 Dispersion 1, or any of 1.1-1.70, wherein the dispersion is pourable (e.g., viscosity of less than 5 Pa*s) or heavy pourable (e.g., viscosity of greater than 5 and less than 20 Pa*s) after high-speed mixing and 1 to 3 passes on a roll mill, preferably pourable (e.g., preferably 0.5 to 5 Pa*s).
1.72 Dispersion, 1 or any of 1.1-1.71, wherein the dispersion has a peak hold viscosity of less than 20 Pa*s at 20/s, e.g., less than 18, or less than 15, or less than 12, or less than 10, or less than 8, or less than 6, or less than 5, or less than 4, or less than 3, or less than 2, or less than 1, or less than 0.5, Pa*S at, at 20/s.
1.73 Dispersion 1, or any of 1.1-1.72, wherein the dispersion is not a shear-thinning dispersion.

It is understood that the compositions according to Dispersion 1 et seq., described herein, may comprise a mixture of discrete polymers according to Formula I which vary in the precise value of the integer n. Thus, in some embodiments, the Dispersion 1 et seq. may comprise only a single polymer according to Formula I (with particular groups R²) having a single value for the integer n. In other embodiments, the Dispersion1 et seq. is understood to comprise a mixture of compounds of Formula I (with particular groups R²) having different values for the integer n, e.g., at least two different compounds of Formula I having different values for the integer n (for example, a mixture which comprises a compound of Formula I wherein n is 0, and a compound of Formula I wherein n is 1). Typically, in such dispersions, substantially all polymers according to Formula I in the composition will have the same groups R², that is, the various polymers according to Formula I in the composition will differ only in the value of the integer n. As used in the preceding sentence (and analogously elsewhere herein), the term “substantially all polymers according to Formula I” is understood to recognize that minor synthetic impurities may be present in which R¹ and/or R² differ from that of the bulk of the composition (e.g., owing to minor impurities in starting materials, minor side-products in the synthesis, or minor amounts of unreacted intermediates, which may be present despite efforts at purification).

Any of the aforementioned compositions may further comprise any one or more cosmetically acceptable ingredients. Such additional ingredients may include one or more of dispersants, surfactants, emulsifiers, emollients, humectants, preservatives, stabilizers, rheological additives, antioxidants, thickeners, and hydrocarbons. In some embodiments, the dispersions may be free of one or more of such categories of ingredients, or the dispersions may be free of all such additional ingredients. It is understood that, while a compound of Formula I may have the properties of, or function as, a dispersant, a surfactant, an emollient, an emulsifier, a stabilizer, a thickener, etc., a composition which is said to be free of such additional ingredients or having a specified amount of such additional ingredients, this is not in reference to the compound(s) of Formula I.

The pigment dispersions according to the present disclosure provide one or more of the following benefits:
A solution to the need for a natural carrier-based pigment dispersion for the personal care and cosmetics markets using untreated, uncoated pigments.
A natural carrier-based dispersion which will eliminate the need for silicones in personal care and cosmetics products.
A pigment dispersion for personal care and cosmetics which will reduce the need for processing time before attaining the desired particle size.
A pigment dispersion for personal care and cosmetics which will require reduced operator time and effort in the form of a shorter pigment wet-out time while under agitation or while mixing.
A pigment dispersion for personal care and cosmetics which will reduce the amount of pigment dispersion required in the final finished product by increasing color strength while maintaining a processable viscosity.
A pigment dispersion for personal care and cosmetics which will increase the processibility and improve the handling of the pigment dispersion, so as to reduce operator time and reduce strain on machinery by lowering the viscosity of the pigment dispersion as compared to a silicone-based dispersion.
A natural-carrier based pigment dispersion with a high degree of compatibility to other natural solvents, so as to allow for the dispersion to be added into multiple phases of a finished cosmetic or personal-care formulation.

The dispersions of the present disclosure are prepared by combining the compound or compounds of formula I (e.g., polycitronellol) and the dispersant (if applicable) using a high-speed mixer, and then the pigment is added while under agitation. The mixture is then high-speed mixed for a set amount of time, before running through a three-roll mill until the desired grind is achieved. In some embodiments, desired grind is considered achieved once it reaches a 7 on the Hegman scale (˜2.7 microns), and the number of passes required to achieve that grind is recorded.

Suitable pigments according to the present disclosure include, but are not limited to, D&C red 6, D&C red 7, D&C red 21, D&C red 27, D&C red 30, D&C red 33, D&C red 34, red 36, D&C red 40, D&C yellow 5, D&C yellow 6, D&C yellow 10, D&C blue 1, red iron oxide, yellow iron oxide, black iron oxide, brown iron oxide, ultramarine blue, ultramarine pink, ultramarine violet, manganese violet, chromium oxide green, chromium hydroxide green, ferric ammonium ferricyanide/iron blue, titanium dioxide, carbon black, D&C black 2, quinoline yellow lake, black PN lake, Patent Blue V lake, erythrosine lake, Lavanya evelyn lake, Lavanya revolutum, Lavanya zuni, Lavanya decorum, and Lavanya Belmont, or any combination thereof.

In a second aspect, the present disclosure further provides cosmetic and personal care compositions comprising Dispersion 1 et seq., including, but not limited to, soaps (liquid or solid), body washes, skin and hair cleansers, skin creams and lotions (e.g., facial creams and lotions, face oils, eye cream, other anti-wrinkle products), ointments, sunscreens, moisturizers, hair shampoos and/or conditioners, deodorants, antiperspirants, other conditioning products for the hair, skin, and nails (e.g., shampoos, conditioners, hair sprays, hair styling gel, hair mousse), decorative cosmetics (e.g., nail polish, eye liner, mascara, lipstick, foundation, concealer, blush, bronzer, eye shadow, lip liner, lip balm,) and dermocosmetics. In some embodiments, the cosmetic and personal compositions have an NOI of from 0.5 to 1.0, e.g., 0.6 to 1.0, or 0.7 to 1.0, or 0.8 to 1.0, or 0.9 to 1.0.

According to ISO16128, polycitronellol (the compound of Formula I wherein R² is H) has a natural origin index (NOI) of 1, while silicones and other synthetic carriers have a NOI of 0. Finished goods manufacturers of cosmetic and personal care products seeking to include the natural content of their formulations can utilize the present invention to achieve a higher NOI for their finished products. The main limitation of NOI for cosmetic color dispersions of the present invention lies within the pigments themselves. Inorganic pigments, for example, iron oxides, ultramarines, mica, or titanium dioxide, generally hold an NOI of 1. Organic pigments, on the other hand, such as those subject to FDA batch certification, hold an NOI of 0. Different pigments capture different color spaces. For a formulator seeking a specific shade in their final product, inorganic pigments alone may not be satisfactory. Thus, a certain percentage of their formulation must always be allotted for ingredients with NOI equal to 0. For a cosmetic dispersion, it is most helpful if the ingredients can contribute some natural origin content, instead of entirely lending to the synthetic portion of the formula.

The present disclosure allows for the carrier vehicle to contribute a NOI of up to 1.0. Additionally, it allows for the use of untreated pigments, whether inorganic or organic in nature. Many pigment surface treatments are not naturally-derived, and thus lend themselves to the synthetic portion of a finished formula. This is especially true for pigment surface treatments used to compatibilize pigments with the silicone phase of a formulation. Untreated pigments would not contribute that surface treatment, thereby preventing that additional synthetic portion and leaving more room for the final formulation to be of natural origin.

Methods to make the compounds of Formula I described herein are disclosed in US2017/0283553 and W02019/028053, the contents of which are incorporated by reference herein in their entireties. Such polymers can generally be made with high degrees of polymerization in a short period of time by using a resin-bound acid catalyst, such as Amberlyst®, under neat, solvent-free conditions. Amberlyst-type resins are recognized in the art and understood to be macroreticular or cellular resins covalently bonded to sulfonic acid or carboxylic acid groups, preferably sulfonic acid groups. Such polymerizations can be done at or below room temperature, preferably at slightly elevated temperature, between 30 and 110° C., or even more preferably between 40 and 90° C. (e.g., about 50° C.). Such polymerizations can take place in batch reactors, semi-batch reactors, or even more preferably in continuous packed bed-type reactors of the type described in International Application PCT/US2017/50808 and US 2019/0210948, the contents of each of which are incorporated herein by reference.

Other aspects regarding the use of compounds and compositions of the present disclosure may be found as disclosed in US2017/0283553 and US2020/0165383, the contents of which are incorporated by reference herein in their entireties.

Unless otherwise indicated, it is to be understood that the terminology used herein is for the purpose of describing particular embodiments only and is not intended to be limiting. In this specification and in the claims that follow, reference will be made to a number of terms, which shall be defined to have the definitions set forth below.

As used herein, the singular forms “a,” “an,” and “the” include plural referents unless the context clearly dictates otherwise. Thus, for example, reference to “a reactant” includes not only a single reactant but also a combination or mixture of two or more different reactant, reference to “a substituent” includes a single substituent as well as two or more substituents, and the like.

As used herein, the phrases “for example,” “for instance,” “such as,” or “including” are meant to introduce examples that further clarify more general subject matter. These examples are provided only as an aid for understanding the disclosure, and are not meant to be limiting in any fashion. Furthermore, as used herein, the terms “may,” “optional,” “optionally,” or “may optionally” mean that the subsequently described circumstance may or may not occur, so that the description includes instances where the circumstance occurs and instances where it does not. For example, the phrase “optionally present” means that an object may or may not be present, and, thus, the description includes instances wherein the object is present and instances wherein the object is not present.

As used herein, the phrase “having the formula” or “having the structure” is not intended to be limiting and is used in the same way that the term “comprising” is commonly used.

In some formulae of the present application, one or more chiral centers are identified by an asterisk placed next to the chiral carbon. In other formulae, no chiral center is identified, but it would be apparent to the skilled artisan that a chiral center is present. In such cases, each chiral isomer is nonetheless covered by these formulae.

Some compounds of the present invention can exist in a tautomeric form which is also intended to be encompassed within the scope of the present invention.

“Tautomers” refers to compounds whose structures differ markedly in arrangement of atoms, but which exist in easy and rapid equilibrium. It is to be understood that the compounds of the invention may be depicted as different tautomers. it should also be understood that when compounds have tautomeric forms, ail tautomeric forms are intended to be within the scope of the invention, and the naming of the compounds does not exclude any tautomeric form. Further, even though one tautomer may be described, the present invention includes all tautomers of the present compounds.

As used herein, the term “salt” can include acid addition salts including hydrochlorides, hydrobromides, phosphates, sulfates, hydrogen sulfates, alkylsulfonates, arylsulfonates, acetates, benzoates, citrates, maleates, fumarates, succinates, lactates, and tartrates; alkali metal cations such as Na⁺, K⁺, Li+, alkali earth metal salts such as Mg²⁺ or Ca²⁺, or organic amine salts, or organic phosphonium salts.

The term “alkyl” as used herein refers to a monovalent or bivalent, branched or unbranched saturated hydrocarbon group having from 1 to 22 carbon atoms, typically although, not necessarily, containing 1 to about 12 carbon atoms, such as methyl, ethyl, n-propyl, isopropyl, n-butyl, isobutyl, t-butyl, pentyl, hexyl, heptyl, octyl, nonyl, decyl, undecyl, dodecyl, and any isomers thereof.

Where a range is recited, such as 0-10 or 1-7, the range embraces all integer values within the range, as well as integer subranges. Thus, the range 0-10 includes 0-9, 0-8, 0-7, 0-6, 0-5, 0-4, 0-3, 0-2, 1-10, 1-9, 1-8, 1-7, 1-6, 1-5, 1-4, 1-3, 1-2, 1-9, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 4-4, 4-3, 5-10, 5- 9, 5-8, 5-7, 5-6, 6-10, 6-9, 6-8, 6-7, 7-10, 7-9, 7-8, 8-10, 8-9, 9-10, 0, 1, 2, 3, 4, 5, 6, 7, 8, 9 and 10.

All percentages used herein, unless otherwise indicated, are by volume.

All ratios used herein, unless otherwise indicated, are by molarity.

Having been generally described herein, the following nonlimiting examples are provided to further illustrate the invention of the present disclosure.

### EXAMPLES

Citronellol polymers have been previously disclosed, such as, in US 2017/0283553, US 2020/0165383, and US 2021/0230364, the contents of each of which are incorporated herein by reference. As used hereinbelow, the term “polycitronellol” refers to polymers having the structure:

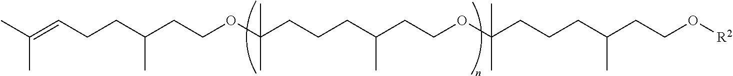

wherein R² is H and n is from 0 to 8 (e.g., 1 to 7). The following examples provide exemplary compositions made according to the present disclosure.

### Example 1: Pigment Wet-Out Time

A series of pigment dispersions are prepared by suspending or dispersing the indicated pigment into the indicated carrier. The silicone polymer Dimethicone 350 cP and the natural oil castor oil are used as comparative carriers. Some dispersions include a dispersant selected from polyhydroxystearic acid (PHSA), PEG-5laurate (P5L), PEG-14laurate (P14L), and bis-hydroxyethyl aminoethyl maleated soybean oil (MSO). The tested dispersions are shown in Table A below (all percentages are percent by weight of the dispersion):

TABLE A

| Formula | Pigment | Carrier | Dispersant |
|---|---|---|---|
| A1 | Titanium dioxide, 60% | Polycitronellol, 34% | PHSA, 6% |
| A2 | Titanium dioxide, 60% | Dimethicone 350 cP, 34% | PHSA, 6% |
| A3 | Titanium dioxide, 60% | Dimethicone 350 cP, 40% | none |
| A4 | Titanium dioxide, 60% | Dimethicone 350 cP, 34% | P5L, 6% |
| A5 | Titanium dioxide, 60% | Dimethicone 350 cP, 34% | P14L, 6% |
| A6 | Titanium dioxide, 60% | Castor oil, 34% | PHSA, 6% |
| B1 | Yellow iron oxide, 61% | Polycitronellol, 39% | None |
| B2 | Yellow iron oxide, 61% | Dimethicone 350 cP, 39% | None |
| B3 | Yellow iron oxide, 61% | Castor oil, 39% | None |
| C1 | Red iron oxide, 61% | Polycitronellol, 36.6% | PHSA, 2.4% |
| C2 | Red iron oxide, 61% | Dimethicone 350cP, 36.6% | PHSA, 2.4% |
| C3 | Red iron oxide, 61% | Dimethicone 350 cP, 39% | None |
| C4 | Red iron oxide, 61% | Castor oil, 36.6% | PHSA, 2.4% |
| D1 | Black iron oxide, 60% | Polycitronellol, 37.6% | MSO, 2.4% |
| D2 | Black iron oxide, 60% | Dimethicone 350 cP, 37.6% | MSO, 2.4% |
| D3 | Black iron oxide, 60% | Dimethicone 350 cP, 40% | None |
| D4 | Black iron oxide, 60% | Castor oil, 37.6% | PHSA, 2.4% |

Pigment wet-out time is measured and reporting in Table 1 below.

TABLE 1

| | | Pigment Wet Out Time |
|---|---|---|
| | | (approximate seconds) |
| Pigment | Titanium Dioxide | |
| Carrier | A1 (Polycitronellol/PHSA) | 43 |
| | A2 (Dimethicone/PHSA) | 172 |
| | A3 (Dimethicone) | 165 |
| | A4 (Dimethicone/P5L) | 150 |
| | A5 (Dimethicone/P14L) | 180 |
| | A6 (Castor Oil/PHSA) | 35 |
| Pigment | Yellow Iron Oxide | |
| Carrier | B1 (Polycitronellol/PHSA) | 150 |
| | B2 (Dimethicone) | 960 |
| | B3 (Castor Oil) | 90 |
| Pigment | Red Iron Oxide | |
| Carrier | C1 (Polycitronellol/PHSA) | 23 |
| | C2 (Dimethicone/PHSA) | 85 |
| | C3 (Dimethicone) | 257 |
| | C4 (Castor Oil/PHSA) | 25 |
| Pigment | Black Iron Oxide | |
| Carrier | D1 (Polycitronellol/MSO) | 30 |
| | D2 (Dimethicone/MSO) | 45 |
| | D3 (Dimethicone) | 150 |
| | D4 (Castor Oil/MSO) | 30 |

These results demonstrate that a polycitronellol polymer offers better pigment-wetting properties as compared to dimethicone-based dispersions, regardless of the inclusion or exclusion of dispersant, or the dispersant type. With regards to castor oil, another natural oil-based carrier, polycitronellol shows similar wet-out properties. This holds the advantage of reduced operator cost, as the pigment can be wetted out faster with less need for monitoring. Operators are free to let the dispersion mix after the pigment is wetted out, which allows for greater overall production speed. In addition, this can hold safety advantages. Pigments can be dusty. By reducing wet-out time, the time spent pouring pigment is reduced, which allows for a less dusty manufacturing environment and cleaner air. The risk of losing pigment via dusting is also reduced, which creates more consistency batch-to-batch and prevents quality issues, both of which can reduce manufacturing costs even further.

### Example 2: Manufacturing Time

Tables 2 and 3 below display data as to the initial high-speed mixing of the pigment with the carrier, with or without dispersant. Tests are performed for each pigment dispersed in polycitronellol carrier, as compared to a formula with either dimethicone 350 cP fluid or castor oil as carrier. In addition, tests are performed using titanium dioxide dispersions with dimethicone 350 cP fluid as the carrier with one of two silicone-compatible dispersants.

High speed mixing is conducted using overhead air mixers and cowles blades. The total high-speed time was set for 1 hour. However, due to the paste-like viscosity of some of tested dispersions, not all dispersions were able to adequately mix. An adequate mix, by this definition, requires a vortex to be created from the top of the cowles blade to the surface of the dispersion. The vortex must pull material from the outer corners of the container without a break in flow. If the dispersion is unable to adequately mix for 1 hour, it is mixed at least until the pigment is wet-out and all raw materials are fully homogenized.

Grind is measured after high-speed mixing using a Hegman grind gauge. The viscosity of the dispersion prior to milling, but after high-speed mixing, is assessed quantitatively using one of the following descriptors in order of increasing viscosity: pourable, heavy pourable, light cream, thick cream, paste, hard paste. Milling is conducted using a Torrey Hills model T65 three roll lab mill. Roller gaps are adjusted according to the individual needs of the dispersion, generally starting with a wider gap distance, and decreasing the gap after subsequent passes.

TABLE 2
High Speed Shearing of Dispersion

| | | Grind after 1 hr | Total High Speed | |
|---|---|---|---|---|
| | | of High Speed | Time | |
| Pigment | Titanium Dioxide | | | |
| Carrier | A1 (Polycitronellol/PHSA) | 6 | 1 | HR |
| | A2 (Dimethicone/PHSA) | 4 | 3 | mins |
| | A3 (Dimethicone) | 6.25 | 1 | HR |
| | A4 (Dimethicone/P5L) | 0 | 3 | mins |
| | A5 (Dimethicone/P14L) | 0 | 3 | mins |
| | A6 (Castor Oil/PHSA) | 6 | 1 | HR |
| Pigment | Yellow Iron Oxide | | | |
| Carrier | B1 (Polycitronellol/PHSA) | 5 | 1 | HR |
| | B2 (Dimethicone) | 0 | 16 | mins |
| | B3 (Castor Oil) | 5.5 | 1 | HR |
| Pigment | Red Iron Oxide | | | |
| Carrier | C1 (Polycitronellol/PHSA) | 6 | 1 | HR |
| | C2 (Dimethicone/PHSA) | 4 | 3 | mins |
| | C3 (Dimethicone) | 5 | 5 | mins |
| | C4 (Castor Oil/PHSA) | 6 | 1 | HR |
| Pigment | Black Iron Oxide | | | |
| Carrier | D1 (Polycitronellol/MSO) | 4 | 1 | HR |
| | D2 (Dimethicone/MSO) | 0 | 1 | HR |
| | D3 (Dimethicone) | 3 | 1 | HR |
| | D4 (Castor Oil/MSO) | 3 | 1 | HR |

TABLE 3
Roll-Milling of Dispersions and Processing Viscosity

| | | Description of | Number of |
|---|---|---|---|
| | | Viscosity after | Passes on Roll |
| | | High Speed | Mill |
| Pigment | Titanium Dioxide | | |
| Carrier | A1 (Polycitronellol/PHSA) | Pourable | 1 |
| | A2 (Dimethicone/PHSA) | Paste | 7 |
| | A3 (Dimethicone) | Heavy pourable | 5 |
| | A4 (Dimethicone/P5L) | Paste | 2 |
| | A5 (Dimethicone/P14L) | Paste | 3 |
| | A6 (Castor Oil/PHSA) | Light cream | 3 |
| Pigment | Yellow Iron Oxide | | |
| Carrier | B1 (Polycitronellol/PHSA) | Heavy pourable | 3 |
| | B2 (Dimethicone) | Hard paste | 9 |
| | B3 (Castor Oil) | Paste | 3 |
| Pigment | Red Iron Oxide | | |
| Carrier | C1 (Polycitronellol/PHSA) | Pourable | 2 |
| | C2 (Dimethicone/PHSA) | Paste | 7 |
| | C3 (Dimethicone) | Paste | 5 |
| | C4 (Castor Oil/PHSA) | Light cream | 2 |
| Pigment | Black Iron Oxide | | |
| Carrier | D1 (Polycitronellol/MSO) | Pourable | 2 |
| | D2 (Dimethicone/MSO) | Light cream | 9 |
| | D3 (Dimethicone) | Heavy pourable | 2 |
| | D4 (Castor Oil/MSO) | Light cream | 1 |

The results demonstrate that polycitronellol is a more ideal grinding vehicle for cosmetic pigment dispersions compared to the other carriers.

The results demonstrate that dispersions using polycitronellol as a carrier, as opposed to either castor oil or dimethicone, offer advantages for a reduced processing viscosity. This prevents manufacturing back-up, because polycitronellol dispersions are demonstrated to be pourable or pumpable. Dimethicone dispersions tend to be much thicker and paste-like, which creates difficulty in handling these materials.

The results demonstrate that dispersions using polycitronellol as a carrier, as opposed to either castor oil or dimethicone, offer advantages regarding milling and milling time. Three-roll milling can be both a labor and energy-intensive process. The mill must be monitored for gap distance, roller speed, proper coverage of the rollers, temperature, etc. Each pass on a roll mill represents a significant operator and manufacturing cost. High speed mixing, on the other hand, is significantly less labor-intensive. While mixers must be monitored for time and temperature, they generally run on their own. It is thus more cost-effective to achieve maximum grind using a high-speed mixer before transferring the dispersion to a roll mill. The results demonstrate that dispersions using polycitronellol as a carrier can adequately run at high speed for up to 1 hour. In addition, the polycitronellol dispersions achieve relatively small grinds compared to dimethicone dispersions, and similar grinds compared to castor oil dispersions, after high-speed shear.

Furthermore, the results demonstrate that, likely due to the points made above, dispersions using polycitronellol as a carrier, as opposed to either castor oil or dimethicone, can achieve a Hegman grind of 7 or more with fewer passes on the roll mill. Dispersions in dimethicone tend to take significantly more passes, and dispersions in castor oil tend take a similar number of passes. Each pass on the roll mill represents a failed grind check, re-loading of material onto the roll mill or into the hopper, re-setting of roller gap distances, re-setting of roller speeds, and re-running of material. Therefore, each pass holds a significant manufacturing cost, as well as the opportunity cost both of running a different product and operator time. By decreasing the number of required passes, the overall manufacturing time is reduced.

### Example 3: Color Loading and Viscosity

Table 4 below displays data as to the dispersion viscosity of pigment dispersions with or without dispersant. Tests are performed for each pigment dispersed in polycitronellol carrier, as compared to a formula with either dimethicone 350 cP fluid or castor oil as carrier. In addition, tests are performed using titanium dioxide dispersions with dimethicone 350 cP fluid as the carrier with one of two silicone-compatible dispersants. Table 4 displays the final viscosity of the dispersions as both a qualitative assessment using one of the following descriptors in order of increasing viscosity: pourable, heavy pourable, light cream, thick cream, paste, hard paste; as well as quantitatively using a TA Discover HR-10 Rheometer. Viscosities measured using the rheometer are determined using both a Peak Hold and Flow Sweep Test under the following parameters:
Peak Hold: 25° C., 60.0 s, 20.01/s
Flow Sweep: 25° C., 0.11/s to 100.01/s

In this way, viscosity is recorded approximately as the plateau of the peak hold curve, or, in case of a linear output, the last recorded viscosity. Flow Sweep testing is used to confirm the peak hold result. For shear-thinning dispersions, the peak hold viscosity is given as a range from the first to last result of the Peak Hold testing.

TABLE 4
Dispersion Viscosities

| | | | Description of |
|---|---|---|---|
| | | Peak Hold | Viscosity of |
| | | Viscosity | Finished |
| | | (Pa*s) at 20/s | Dispersion |
| Pigment | Titanium Dioxide | | |
| Carrier | A1 (Polycitronellol/PHSA) | 3.97 | Pourable |
| | A2 (Dimethicone/PHSA) | 240.1-115.06 | Hard paste |
| | A3 (Dimethicone) | 24.51-18.20 | Light cream |
| | A4 (Dimethicone/P5L) | 337.69-213.32 | Paste |
| | A5 (Dimethicone/P14L) | 447.74-52.23 | Paste |
| | A6 (Castor Oil/PHSA) | 10.33 | Light Cream |
| Pigment | Yellow Iron Oxide | | |
| Carrier | B1 (Polycitronellol/PHSA) | 16.81 | Heavy pourable |
| | B2 (Dimethicone) | 68.35-21.42 | Light Cream |
| | B3 (Castor Oil) | 34.65 | Light Cream |
| Pigment | Red Iron Oxide | | |
| Carrier | C1 (Polycitronellol/PHSA) | 1.48 | Pourable |
| | C2 (Dimethicone/PHSA) | 56.91-33.63 | Thick cream |
| | C3 (Dimethicone) | 31.61-26.56 | Light Cream |
| | C4 (Castor Oil/PHSA) | 11.57 | Light Cream |
| Pigment | Black Iron Oxide | | |
| Carrier | D1 (Polycitronellol/MSO) | 5.28 | Heavy pourable |
| | D2 (Dimethicone/MSO) | 46.37-41.67 | Light Cream |
| | D3 (Dimethicone) | 10.02-6.41 | Light Cream |
| | D4 (Castor Oil/MSO) | 10.05 | Light Cream |

The results demonstrate that dispersions using polycitronellol as a carrier are significantly less viscous in comparison to dispersions made with castor oil or dimethicone. This provides manufacturing advantages, since thinner materials are more easily poured or pumped. Paste-like dispersions may clog tubing and pose potential hazards if more power is required to handle them. To achieve a reduced viscosity, the pigment percentage must be reduced and/or dispersant loading must be increased. This is not ideal, however, because in that scenario more dispersion is required to impart the same color to the finished good formulation, and/or additional dispersant raises the cost of the dispersion. This forces the dispersion customer to purchase more raw materials. In either case, the customer must pay more for the material

### Example 4: Color Strength

Relative color strength is evaluated for several dispersions. Tests are performed for each pigment dispersed in polycitronellol carrier, as compared to a formula with either dimethicone 350 cP fluid or castor oil as carrier. In addition, tests are performed using titanium dioxide dispersions with dimethicone 350 cP fluid as the carrier with one of two silicone-compatible dispersants. In each case, the polycitronellol dispersion is named the “standard” and the other dispersions are read relative to their respective standard based on pigment type.

Colors are read in a white-tinted base at 3% dispersion. White dispersions are read in a green-tinted base at 10% dispersion. They are drawn down at equal thickness with respect to their standards, ensuring complete opacity, and read over white cardstock. Because color strength is measure of the dispersions ability to overcome white, for the titanium dioxide dispersions, drawdowns are read backwards. Thus, the charted data is accurate for titanium dioxide and should be read as-is.

TABLE 5
Color Readings of Dispersions

| | | Color | | | | |
|---|---|---|---|---|---|---|
| Pigment | Titanium Dioxide | Strength | DE | DL | Da | Db |
| Carrier | A1 (Polycitronellol/PHSA) | 100 | 0.00 | 0.00 | 0.00 | 0.00 |
| | A2 (Dimethicone/PHSA) | 97.23 | 0.47 | 0.40 | −0.16 | 0.21 |
| | A3 (Dimethicone) | 90.60 | 3.88 | 3.48 | 0.13 | −1.73 |
| | A4 (Dimethicone/P5L) | 106.76 | 1.35 | −0.43 | −1.26 | 0.16 |
| | A5 (Dimethicone/P14L) | 108.29 | 1.28 | −0.65 | −1.09 | 0.17 |
| | A6 (Castor Oil/PHSA) | 107.64 | 1.50 | −0.74 | −1.22 | 0.47 |
| Pigment | Yellow Iron Oxide | | | | | |
| Carrier | B1 (Polycitronellol/PHSA) | 100 | 0.00 | 0.00 | 0.00 | 0.00 |
| | B2 (Dimethicone) | 98.33 | 0.19 | 0.00 | −0.01 | −0.19 |
| | B3 (Castor Oil) | 102.27 | 0.36 | −0.36 | 0.06 | 0.03 |
| Pigment | Red Iron Oxide | | | | | |
| Carrier | C1 (Polycitronellol/PHSA) | 100 | 0.00 | 0.00 | 0.00 | 0.00 |
| | C2 (Dimethicone/PHSA) | 98.73 | 0.24 | 0.17 | −0.16 | 0.06 |
| | C3 (Dimethicone) | 96.57 | 0.89 | 0.17 | −0.39 | −0.78 |
| | C4 (Castor Oil/PHSA) | 101.16 | 0.29 | −0.23 | −0.13 | −0.12 |
| Pigment | Black Iron Oxide | | | | | |
| Carrier | D1 (Polycitronellol/MSO) | 100 | 0.00 | 0.00 | 0.00 | 0.00 |
| | D2 (Dimethicone/MSO) | 93.10 | 1.41 | 1.39 | −0.10 | −0.23 |
| | D3 (Dimethicone) | 99.18 | 0.22 | 0.21 | −0.04 | −0.06 |
| | D4 (Castor Oil/MSO) | 99.21 | 0.08 | 0.06 | 0.03 | 0.04 |

The results demonstrate that dispersions made with polycitronellol have a higher color strength, as opposed to either castor oil or dimethicone, with the exception of titanium dioxide. A color strength of +/−5% is generally accepted as within tolerance. Dispersions made with polycitronellol have an equal or higher color strength as opposed to either castor oil or dimethicone within this tolerance.

Although titanium dioxide has a higher color strength in dimethicone dispersions with an adjusted dispersant than polycitronellol, the dimethicone dispersions are still not optimal or usable due to the high trade-off between the increased color strength and physical properties. The dimethicone dispersions with adjusted dispersants are about 7% higher in tint strength. However, to obtain a viscosity that could qualify as a cream, rather than a paste, the dispersion would have to be let-down with additional silicone at a rate much greater than 7%. In that case, the color strength would actually be weaker than the polycitronellol standard.

### Example 5: Compatibility

To test compatibility, dispersions made with are added to an experimental solvent at a 1:9 ratio and mixed for about 30 seconds using a combination of hand-stirring and high-speed mixing. The mixture is then rested for about 1 minute to allow for the release of trapped air and it is then photographed and examined for various physical properties including homogenization, settling, separation, and color development.

Dispersions made with polycitronellol, as described in the preceding examples, as opposed to either castor oil or dimethicone, are highly compatible with polar solvents such as castor oil and caprylic/capric triglycerides, and with natural nonpolar oils such as mineral oil, jojoba oil, isododecane, alkanes, and squalene. They are somewhat compatible with propylene glycol and nonpolar silicones such as cyclopentasiloxane and dimethicone. It is important to note, for the “incompatible” solvents, that the dispersions of the present disclosure are still compatible with silicones in a finished good formula, so long as they are added in different phases or otherwise are not expected to solubilize one another. In finished formulas, no incompatibility has so far been recognized.

I/We claim:

1. A pigment dispersion comprising a pigment dispersed in a grinding vehicle and/or carrier, wherein the grinding vehicle and/or carrier comprises a compound according to Formula I below: 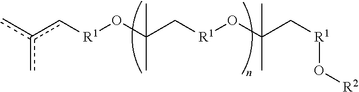
wherein R¹ is CH₂CH₂CH(CH₃)CH₂CH₂, R² is H, C₁₋₂₀alkyl (e.g., C₁₋₃alkyl), or —C(O)—C₁₋₂₀alkyl (e.g., —C(O)—C₁₋₃alkyl); and wherein n is an integer between 0 and 20.

2. The dispersion according to claim 1, wherein in the compound of Formula I, R² is H.

3. The dispersion according to claim 1, wherein in the compound of Formula I, R²is C₁₋₂₀alkyl (e.g., lower alkyl (e.g., C₁₋₆), or C₁₋₁₂).

4. The dispersion according to claim 1, wherein in the compound of Formula I, R²is —C(O)—C₁₋₂₀ alkyl (e.g., —C(O)—C₁₋₆ alkyl).

5. The dispersion according to claim 1, wherein in the compound of Formula I, n is 0 to 10 (e.g., 1 to 9, or 2 to 8, or 1 to 7, or 2 to 6, or 1 to 5, or 2 to 4).

6. The dispersion according to claim 1, wherein in the compound of Formula I, n is 1 to 7 (e.g., 1 to 4, or 2 to 5, or 3 to 6, or 4 to 7, or 2 to 6).

7. The dispersion according to claim 1, wherein in the compound of Formula I, the terminal group 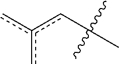
is

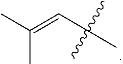 [SMILES: *C=C(C)C]

8. The dispersion according to claim 1, wherein the Compound of Formula I is: 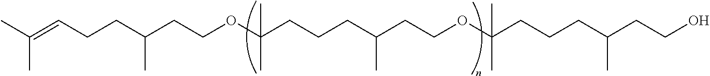
where n: 0-20 (e.g., 0-10, 1-9, 1-7, 1-4, 1-3, 0-5, 0-4, 0-3, 0, 1, 2, 3, 4, or 5).

9. The dispersion according to claim 1, wherein the dispersion is a non-aqueous dispersion.

10. The dispersion according to claim 1, wherein the grinding vehicle and/or carrier further comprises one or more natural or naturally derived oils, e.g., vegetable oils (e.g., castor oil).

11. The dispersion according to claim 1, wherein the grinding vehicle and/or carrier consists of the one or more Compounds of Formula I.

12. The dispersion according to claim 1, wherein the one or more Compounds of Formula I act as a grinding vehicle, as a carrier, or as a grinding vehicle and as a carrier.

13. The dispersion according to claim 1, wherein the pigment is selected from iron oxide (e.g., red iron oxide, yellow iron oxide, black iron oxide or a mixture thereof), titanium dioxide, ultramarines, mica, or any combination thereof.

14. The dispersion according to claim 1, wherein the pigments are untreated and/or uncoated, and optionally wherein the pigments are cosmetic-grade.

15. The dispersion according to claim 1, wherein the dispersion further comprises a dispersant.

16. The dispersion according to claim 1, wherein the dispersion does not comprise any silicone polymer.

17. The dispersion according to claim 1, wherein the dispersion consists essentially of the pigment or pigments, the compound or compounds of Formula I, and optionally the dispersant.

18. The dispersion according to claim 1, wherein the dispersion comprises 40% or more of the pigment or pigments, by weight of the dispersion, e.g., 50% or more, 60% or more, or 70% or more, or 80% or more, or 90% or more, or 95% or more.

19. The dispersion according to claim 1, wherein the dispersion comprises 50% or less of the compound or compounds of Formula I, by weight of the dispersion, e.g., 40% or less, or 30% or less, or 20% or less, or 10% or less, or 5% or less.

20. The dispersion according to claim 1, wherein the dispersion comprises not more than 25% by weight of any dispersant(s) or other components (other than the pigment(s) and the compound(s) of Formula I), by weight of the dispersion, e.g., not more than 20%, or not more than 15%, or not more than 10%, or not more than 5%, or not more than 4%, or not more than 3%, or not more than 2%, or not more than 1%.

21. A cosmetic or personal care compositions comprising a dispersion according to claim 1.

22. The cosmetic or personal care composition according to claim 21, wherein the composition has a natural origin index (NOI) of from 0.5 to 1.0, e.g., 0.6 to 1.0, or 0.7 to 1.0, or 0.8 to 1.0, or 0.9 to 1.0.
